SEQUENCE LISTING (33 sequences)

SEQ ID NO: 1 - DNA, 21 bp, Homo sapiens
ctccaccatgccctctacaca

SEQ ID NO: 2 - DNA, 21 bp, Homo sapiens
acatgatcaactgggcgaaga

SEQ ID NO: 3 - DNA, 21 bp, Homo sapiens
tccctgccaatatcttgggta

SEQ ID NO: 4 - DNA, 20 bp, Homo sapiens
agttgtgtcgagctcacagc

SEQ ID NO: 5 - DNA, 21 bp, Homo sapiens
tgagcagatgttccatgccct

SEQ ID NO: 6 - DNA, 20 bp, Homo sapiens
tccagtatgtaccctctggt

SEQ ID NO: 7 - DNA, 20 bp, Homo sapiens
gagccagcccgaagtctgta

SEQ ID NO: 8 - DNA, 21 bp, Homo sapiens
tcttagaccatgtccgggaaa

SEQ ID NO: 9 - DNA, 20 bp, Homo sapiens
gatcctgatgattggtctcg

SEQ ID NO: 10 - DNA, 20 bp, Homo sapiens
cgggcactgagtgttgaatt

SEQ ID NO: 11 - DNA, 20 bp, Homo sapiens
gatgctttggtttgggtgat

SEQ ID NO: 12 - DNA, 20 bp, Homo sapiens
ggaggatgtgcggctcgtac

SEQ ID NO: 13 - DNA, 25 bp, Homo sapiens
tacaggccaaattcagataatcgac

SEQ ID NO: 14 - DNA, 21 bp, Homo sapiens
ggaaccgagatgatgtagcca

SEQ ID NO: 15 - DNA, 22 bp, Homo sapiens
tgacaagtcttaatcaactagg

SEQ ID NO: 16 - DNA, 23 bp, Homo sapiens
tcactttttatgaaagagaaggg

SEQ ID NO: 17 - DNA, 18 bp, Homo sapiens
gccgccccatgtgctgat

SEQ ID NO: 18 - DNA, 21 bp, Homo sapiens
ggaccccgtgatggaggactt

SEQ ID NO: 19 - DNA, 25 bp, Homo sapiens
tggttggcattctgctggtcgtggt

SEQ ID NO: 20 - DNA, 25 bp, Homo sapiens
tggccgacattcagagtcaatcatc

SEQ ID NO: 21 - DNA, 52 bp, Homo sapiens
aattctaatacgactcactatagggagaaggctccaccatgccctctacaca

SEQ ID NO: 22 - DNA, 52 bp, Homo sapiens
aattctaatacgactcactatagggagaaggtccctgccaatatcttgggta

SEQ ID NO: 23 - DNA, 52 bp, Homo sapiens
aattctaatacgactcactatagggagaaggtgagcagatgttccatgccct

SEQ ID NO: 24 - DNA, 51 bp, Homo sapiens
aattctaatacgactcactatagggagaagggagccagcccgaagtctgta

SEQ ID NO: 25 - DNA, 20 bp, Homo sapiens
acatgaaggtgctccttgcc

SEQ ID NO: 26 - DNA, 20 bp, Homo sapiens
gtccctgtgccctactcctt

SEQ ID NO: 27 - DNA, 22 bp, Homo sapiens
caggctgtcttgactgtcgtga

SEQ ID NO: 28 - DNA, 20 bp, Homo sapiens
aggagagaaaggatttggct

SEQ ID NO: 29 - DNA, 20 bp, Homo sapiens
gatccagggcggagacttca

SEQ ID NO: 30 - DNA, 53 bp, Homo sapiens
aattctaatacgactcactatagggagaaggcaggctgtcttgactgtcgtga

SEQ ID NO: 31 - DNA, 32 bp, Homo sapiens
cgatcggatcctgatgattggtctcgcgatcg

SEQ ID NO: 32 - DNA, 32 bp, Homo sapiens
cgatcgcgggcactgagtgttgaattcgatcg

SEQ ID NO: 33 - DNA, 32 bp, Homo sapiens
cgatcggatccagggcggagacttcacgatcg

(19) United States
(12) Patent Application Publication
(10) Pub. No.: US 2007/0134669 A1
(43) Pub. Date: Jun. 14, 2007

(54) Method for diagnosis/prognosis of breast cancer

(75) Inventor: Thibaut Verjat, Lyon (FR)

Correspondence Address: OLIFF & BERRIDGE, PLC, P.O. BOX 19928, ALEXANDRIA, VA, 22320, US

(73) Assignee: bioMerieux, Marcy l'Etoile (FR)

(21) Appl. No.: 10/579,986

(22) PCT Filed: Dec. 7, 2004
(86) PCT No.: PCT/FR04/50661

§ 371 Date: Sep. 19, 2006

(30) Foreign Application Priority Data
Dec. 9, 2003 (FR) 0314364

(51) Int. Cl. C12Q 1/68; C12P 19/34

(52) U.S. Cl. 435/6; 435/91.2

(57) ABSTRACT

The present invention relates to a method for diagnosis/prognosis of breast cancer including the following stages: A—the nuclear material is extracted from a biological specimen, B—at least one pair of amplification primers is used for obtaining amplicons of at least one target sequence of the nuclear material, C—at least one detection probe is used for detecting the presence of said amplicons. In stage B, the pair of primers includes at least one amplification primer including at least 10 nucleotide motifs of a nucleotide sequence selected from SEQ ID No. 1 to SEQ ID No. 24 and/or in stage C, the detection probe includes at least 10 nucleotide motifs of a nucleotide sequence selected from SEQ ID No. 1 to SEQ ID No. 20. The invention also relates to amplification primers and hybridization probes which can be employed in this method, as well as a kit for diagnosis/prognosis of breast cancer.

The present invention relates to a method for diagnosis/prognosis of breast cancer. The invention also relates to amplification primers and hybridization probes which can be employed in this method, as well as a kit for diagnosis/prognosis of breast cancer.

Breast cancer is a commonly occurring disease: one woman out of eleven develops breast cancer in the course of her life. However, because there are various types of breast cancers, and varying prognosis of breast cancer, women affected by it do not all follow the same treatment: the doctor offers each patient a treatment appropriate to her situation, in order to obtain the best chances of a cure.

Thus, hormonotherapy, which is a systemic treatment in breast cancers, is used in hormone-dependent breast cancers, i.e. in the case of tumors expressing hormone receptors on the surface of their cells. Post-operatively, hormonotherapy can be used alone or alternating with adjuvant chemotherapy. In recurrence of the disease, hormonotherapy can be prescribed either alone, or combined or alternating with chemotherapy.

Chemotherapy, in itself, is a systemic cancer treatment since the drugs, carried by the blood circulation, are able to act everywhere in the body. Chemotherapy has an important place in the therapeutic arsenal, especially in the last ten years or so, with the appearance of novel molecules. The drugs are most often administered by intravenous, subcutaneous, or intramuscular perfusion.

Thus, treatment may or may not be oriented towards hormonotherapy, depending on expression of hormone receptors on the surface of the hormone-secreting cells.

We may mention notably the estrogen receptors ESR1 and ESR2 and the progesterone receptor (PGR), which are the best-known parameters for predicting the response to hormonotherapy in breast cancer. Thus the content of ESR1 is used as a prognostic indicator, and for predicting a patient's response to treatment with antiestrogens, such as Tamoxifen® (Osborne C et al., Breast Cancer Res treat 51:227-238, 1998; Goldhirsch et al., J Clin Oncol 19:3817-3827, 2001). The presence of the PGR receptor is also used for monitoring hormonotherapy, and as a prognostic marker (Horwitz et al., Recent Prog Horm Res 41: 249-316, 1995). We may also mention the HER2 receptor, which is said to be overexpressed in about ¼ of invasive breast cancers (Slamon et al., Science, 1987, 235: 177-182)

In order to offer patients appropriate treatment, it is therefore essential to know the expression of genes coding for hormone receptors, such as ESR1, ESR2, HER2 and PGR. This expression is investigated most often on the primary tumor and most often by immunohistochemistry. In doubtful cases, investigation of gene amplification by in situ hybridization (FISH) is the method of reference, notably in the case of HER2. For some years, it has been possible to detect small tumors, permitting early diagnosis of breast cancer, but prognosis of this cancer is still difficult from the small amount of tumoral tissue, which makes protein quantification of the aforementioned hormone receptors difficult. The techniques of molecular biology then become indispensable for the quantification of hormone receptors, as they require smaller amounts of tumoral tissue (Fuqua et al., Natl Cancer Inst 82: 859-861, 1997; Fasco et al., Anal Biochem, 245: 167-178, 1997; Poola et al., Anal Biochem, 258: 209-215, 1998).

The present invention proposes a novel method for diagnosis/prognosis of breast cancer. This method notably employs novel nucleotide sequences which can be used either as amplification primers or as hybridization probes. The method according to the invention notably makes it possible to determine the most suitable treatment for a patient with breast cancer.

Thus, the invention relates to a method for diagnosis/prognosis of breast cancer comprising the following stages:
A—the nuclear material is extracted from a biological specimen,
B—at least one pair of amplification primers is used to obtain amplicons of at least one target sequence of the nuclear material
C—at least one detection probe is used for detecting the presence of said amplicons
   characterized in that, in stage B, said pair of primers comprises at least one amplification primer comprising at least 10 nucleotide motifs of a nucleotide sequence selected from SEQ ID No. 1 to SEQ ID No. 24 and/or in stage C, said detection probe comprises at least 10 nucleotide motifs of a nucleotide sequence selected from SEQ ID No. 1 to SEQ ID No. 20.

Surprisingly, the inventors thus discovered that the use, in a method for diagnosis/prognosis of breast cancer, of a nucleotide sequence comprising at least 10 nucleotide motifs of a nucleotide sequence selected from SEQ ID No. 1 to SEQ ID No. 20 is very suitable as amplification primer for amplifying target sequences, such as the gene coding for ESR1, ESR2, PGR or HER2. The inventors also discovered that the use of a nucleotide sequence comprising at least 10 nucleotide motifs of a nucleotide sequence selected from SEQ ID No. 1 to SEQ ID No. 20 as hybridization probe is very suitable for specific hybridization on target sequences, such as the genes coding for ESR1, ESR2, PGR or HER2.

In the sense of the present invention, biological specimen means any specimen that may contain a nuclear material as defined hereafter. This biological specimen can be taken from a patient and can notably be a specimen of tissues, of blood, of serum, of saliva or of circulating cells obtained from the patient. Preferably, this biological specimen is taken from a tumor. This biological specimen is obtained in any manner known by a person skilled in the art.

In the sense of the present invention, the nuclear material comprises a sequence of nucleic acids such as a sequence of deoxyribonucleic acids (DNA) or of ribonucleic acids (RNA). According to a preferred embodiment of the invention, the nuclear material comprises a sequence of deoxyribonucleic acids. According to a preferred embodiment of the invention, the nuclear material is extracted from a biological specimen taken from a patient.

Nucleotide sequence (or sequences of nucleic acids or nucleotide fragment or oligonucleotide, or polynucleotide) means a chain of nucleotide motifs joined together by phosphate bonds, characterized by the information sequence of the natural nucleic acids, which are able to hybridize with another sequence of nucleic acids, wherein the chain can contain monomers of different structures and can be obtained from a natural nucleic acid molecule and/or by genetic recombination and/or by chemical synthesis.

Nucleotide motif means a derivative of a monomer, which can be a natural nucleotide of nucleic acid whose constituent elements are a sugar, a phosphate group and a nitrogenous base; in DNA the sugar is deoxy-2-ribose, in RNA the sugar is ribose; depending on whether it is a matter of DNA or RNA, the nitrogenous base is selected from adenine, guanine, uracil, cytosine or thymine; or alternatively the monomer is a nucleotide modified in at least one of the three constituent elements; as an example, modification can occur either at the level of the bases, with modified bases such as inosine, methyl-5-deoxycytidine, deoxyuridine, dimethylamino-5-deoxyuridine, diamino-2,6-purine, bromo-5-deoxyuridine or any other modified base capable of hybridization, either at the level of the sugar, for example the replacement of at least one deoxyribose by a polyamide (P. E. Nielsen et al., Science, 254, 1497-1500 (1991), or at the level of the phosphate group, for example its replacement by esters notably selected from the diphosphates, alkyl- and arylphosphonates and phosphorothioates. This nuclear material comprises at least one target sequence. By target sequence. we mean a sequence in which the chain of nucleotide motifs is specific to a target gene, such as preferably the gene coding for ESR1, ESR2, PGR or HER2. According to a preferred embodiment of the invention, the target sequence is comprised in a gene selected from the genes coding for ESR1, ESR2, PGR or HER2. Hereinafter, we shall use the term target sequence, whether it is single-stranded or double-stranded.

In stage A, the nuclear material is extracted from a biological specimen by any protocol known by a person skilled in the art. Indicatively, the extraction of nucleic acids can be carried out by a stage of lysis of the cells present in the biological specimen, in order to release the nucleic acids contained in the protein and/or lipid envelopes of the cells (as cellular debris that disturbs subsequent reactions). As an example, it is possible to use the methods of lysis as described in patent applications WO00/05338 using mixed magnetic and mechanical lysis, WO99/53304 using electrical lysis, and WO99/15321 using mechanical lysis.

A person skilled in the art will be able to use other well-known methods of lysis, such as thermal or osmotic shock, or chemical lysis by chaotropic agents such as guanidium salts (U.S. Pat. No. 5,234,809). This lysis stage can also be followed by a purification stage, permitting separation between the nucleic acids and the other cellular constituents salted-out in the lysis stage. This stage generally permits the nucleic acids to be concentrated, and can be adapted to the purification of DNA or of RNA. As an example, it is possible to use magnetic particles optionally coated with oligonucleotides, by adsorption or covalent bonding (cf. U.S. Pat. No. 4,672,040 and U.S. Pat. No. 5,750,338), and thus purify the nucleic acids which become attached to these magnetic particles, in a washing stage. This stage for purification of the nucleic acids is particularly interesting if further amplification of said nucleic acids is desired. A particularly interesting embodiment of these magnetic particles is described in patent applications WO97/45202 and WO99/35500. Another interesting example of a method of purification of nucleic acids is the use of silica, either in the form of a column, or in the form of inert particles (Boom R. et al., J. Clin. Microbiol., 1990, No. 28(3), p. 495-503) or magnetic particles (Merck: MagPrep® Silica, Promega: MagneSil™ Paramagnetic particles). Other methods that are widely used are based on ion-exchange resins in a column or in the form of paramagnetic particles (Whatman: DEAE-Magarose) (Levison P R et al., J. Chromatography, 1998, p. 337-344). Another method that is very appropriate but not exclusive for the invention is adsorption on a metal oxide support (company Xtrana: Xtra-Bind™ matrix).

If we wish to specifically extract the DNA from a biological specimen, extraction can notably be carried out with phenol, with chloroform and with alcohol to remove the proteins and precipitate the DNA with 100% alcohol. The DNA can then be deposited by centrifugation, washed and resuspended.

In stage B, at least one pair of amplification primers is used, to obtain amplicons of at least one target sequence of the nuclear material.

In the sense of the present invention, amplification primer means a nucleic acid sequence comprising from 10 to 100 nucleotide motifs, preferably from 15 to 25 nucleotide motifs. This amplification primer comprises at least 10, preferably 15 and even more preferably 20 nucleotide motifs of a sequence selected from SEQ ID No. 1 to 20. In the sense of the present invention, an amplification primer comprising at least 10, preferably 15 and even more preferably 20 nucleotide motifs of
a sequence homologous to SEQ ID No. 1 to SEQ ID No. 20, i.e.
   the sequence complementary or sufficiently complementary to SEQ ID No. 1 to 20
   a sequence displaying sufficient homology for hybridizing with SEQ ID No. 1 to SEQ ID No. 20 or with the sequence complementary to SEQ ID No. 1 to SEQ ID No. 20,
a sequence comprising a sequence from SEQ ID No. 1 to SEQ ID No. 20 (or a sequence homologous to SEQ ID No. 1 to SEQ ID No. 20 as defined previously) in which uracil bases are substituted for the thymine bases,
   and which would have the same function as the amplification primer according to the invention, i.e. amplification of all or part of the gene coding for ESR1, ESR2, PGR or HER2, is regarded as equivalent to the amplification primer according to the invention.

A pair of amplification primers permits the initiation of enzymatic polymerization, such as notably a reaction of enzymatic amplification.

Reaction of enzymatic amplification means a process that generates multiple copies (or amplicons) of a nucleic acid sequence by the action of at least one enzyme. In the sense of the present invention, amplicons means the copies of the target sequence obtained in a reaction of enzymatic amplification. Such reactions of amplification are familiar to a person skilled in the art and we may mention notably PCR (Polymerase Chain Reaction), as described in U.S. Pat. No. 4,683,195, U.S. Pat. No. 4,683,202 and U.S. Pat. No. 4,800,159; LCR (Ligase Chain Reaction), disclosed for example in patent application EP-A-0 201 184; RCR (Repair Chain Reaction), described in patent application WO-A-90/01069; 3SR (Self Sustained Sequence Replication) with patent application WO-A-90/06995; NASBA (Nucleic Acid Sequence-Based Amplification) with patent application WO-A-91/02818, or TMA (Transcription Mediated Amplification) with U.S. Pat. No. 5,399,491.

As a general rule, these reactions of enzymatic amplification generally employ a succession of cycles comprising the following stages:
denaturation of the target sequence if it is double-stranded in order to obtain two complementary target strands,
hybridization of each of these target strands, obtained in the preceding stage of denaturation, with at least one amplification primer,
formation, on the basis of the amplification primers, of strands complementary to the strands on which they are hybridized in the presence of a polymerase enzyme and a nucleoside triphosphate (ribonucleoside triphosphate and/or deoxyribonucleoside triphosphate depending on the technique employed),
   this cycle being repeated a determined number of times to obtain the target sequence in a sufficient proportion for it to be detectable.

Hybridization means the process in which, in appropriate conditions, two nucleic acid sequences such as notably an amplification primer and a target sequence or a hybridization probe and a target sequence, are joined together by stable and specific hydrogen bonds to form a double strand. These hydrogen bonds are formed between the complementary bases adenine (A) and thymine (T) (or uracil (U)) (called an A-T bond) or between the complementary bases guanine (G) and cytosine (C) (called a G-C bond). The hybridization of two nucleic acid sequences can be complete (they are then called complementary sequences), i.e. the double strand obtained during this hybridization is comprised solely of A-T bonds and C-G bonds. This hybridization can be partial (they are then called sufficiently complementary sequences), i.e. the double strand obtained is comprised of A-T bonds and C-G bonds permitting formation of the double strand, but also comprises bases that are not bound to a complementary base. Hybridization between two complementary sequences or sufficiently complementary sequences depends on the working conditions that are employed, and notably on stringency. Stringency is defined notably in relation to the base composition of the two nucleic acid sequences, as well as by the degree of mismatch between these two nucleic acid sequences. Stringency can also be a function of the parameters of the reaction, such as the concentration and the type of ionic species present in the hybridization solution, the nature and the concentration of denaturing agents and/or the hybridization temperature. All these facts are well known and the appropriate conditions can be determined by a person skilled in the art.

More precisely, NASBA is a technology of isothermal amplification of nucleic acid based on the combined action of three enzymes (reverse transcriptase AMV, RNase-H and polymnerase-RNA T7). Combined with amplification primers specific to a target sequence, it amplifies the RNA targets more than a billion-fold in 90 minutes. The amplification reaction takes place at 41° C. and gives molecules of single-stranded RNA as the final product. NASBA requires a pair of primers, at least one of which comprises a promoter permitting the initiation of transcription by a polymerase of bacteriophage T7. Such a primer is preferably selected from SEQ ID No. 21 to 24. According to a particular embodiment of the invention, said primer pair comprises at least one amplification primer comprising a promoter permitting the initiation of transcription by a polymerase of bacteriophage T7.

According to a particular embodiment of the invention, said pair of primers, used in stage B, is selected from the following pairs of primers:
a first amplification primer comprising at least 10, preferably 15 and even more preferably 20 nucleotide motifs of nucleotide sequence SEQ ID No. 1 and a second amplification primer comprising at least 10, preferably 15 and even more preferably 20 nucleotide motifs of nucleotide sequence SEQ ID No. 2; indicatively, when the first primer has SEQ ID No. 1 as its sequence, and the second primer has SEQ ID No. 2 as its sequence, an amplicon is obtained that is specific to the gene coding for ESR1, with a size of 202 base pairs, which corresponds to sequence 1427-1629 on the sequence of the reference gene coding for ESR1 (Genbank X03635).
a first amplification primer comprising at least 10, preferably 15 and even more preferably 20 nucleotide motifs of nucleotide sequence SEQ ID No. 3 and a second amplification primer comprising at least 10, preferably 15 and even more preferably 20 nucleotide motifs of nucleotide sequence SEQ ID No. 4; indicatively, when the first primer has SEQ ID No. 3 as its sequence, and the second primer has SEQ ID No. 4 as its sequence, an amplicon is then obtained that is specific to the gene coding for PGR, with a size of 184 base pairs, which corresponds to sequence 2761-2945 on the reference sequence coding for PGR (Genbank NM_{—}000926).
a first amplification primer comprising at least 10, preferably 15 and even more preferably 20 nucleotide motifs of nucleotide sequence SEQ ID No. 5 and a second amplification primer comprising at least 10, preferably 15 and even more preferably 20 nucleotide motifs of nucleotide sequence SEQ ID No. 6; indicatively, when the first primer has SEQ ID No. 5 as its sequence, and the second primer has SEQ ID No. 6 as its sequence, an amplicon is then obtained that is specific to the ESR2 gene, with a size of 210 base pairs, which corresponds to sequence 1640-1850 on the reference sequence coding for ESR2 (Genbank MN_{—}001437).
a first amplification primer comprising at least 10, preferably 15 and even more preferably 20 nucleotide motifs of nucleotide sequence SEQ ID No. 7 and a second amplification primer comprising at least 10, preferably 15 and even more preferably 20 nucleotide motifs of nucleotide sequence SEQ ID No. 8; indicatively, when the first primer has SEQ ID No. 7 as its sequence, and the second primer has SEQ ID No. 8 as its sequence, an amplicon is then obtained that is specific to the gene coding for HER2, with a size of 185 base pairs, which corresponds to sequence 2567-2752 on the reference sequence coding for HER2 (Genbank NM_{—}00448).
a first amplification primer comprising at least 10, preferably 15 and even more preferably 20 nucleotide motifs of nucleotide sequence SEQ ID No. 13 and a second amplification primer comprising at least 10, preferably 15 and even more preferably 20 nucleotide motifs of nucleotide sequence SEQ ID No. 14; indicatively, when the first primer has SEQ ID No. 13 as its sequence, and the second primer has SEQ ID No. 14 as its sequence, an amplicon is obtained that is specific to the gene coding for ESR1, with a size of 858 base pairs, which corresponds to sequence 808-1666 on the reference sequence coding for ESR1 (Genbank X03635).
a first amplification primer comprising at least 10, preferably 15 and even more preferably 20 nucleotide motifs of nucleotide sequence SEQ ID No. 15 and a second amplification primer comprising at least 10, preferably 15 and even more preferably 20 nucleotide motifs of nucleotide sequence SEQ ID No. 16; indicatively, when the first primer has SEQ ID No. 15 as its sequence, and the second primer has SEQ ID No. 16 as its sequence, an amplicon is then obtained that is specific to the gene coding for PGR, with a size of 658 base pairs, which corresponds to sequence 2319-2977 on the reference sequence coding for PGR (Genbank NM_{—}000926).
a first amplification primer comprising at least 10, preferably 15 and even more preferably 20 nucleotide motifs of nucleotide sequence SEQ ID No. 17 and a second amplification primer comprising at least 10, preferably 15 and even more preferably 20 nucleotide motifs of nucleotide sequence SEQ ID No. 18; indicatively, when the first primer has SEQ ID No. 17 as its sequence, and the second primer has SEQ ID No. 18 as its sequence, an amplicon is then obtained that is specific to the gene coding for ESR2, with a size of 702 base pairs, which corresponds to sequence 1246-1948 on the reference sequence coding for ESR2 (Genbank MN_{—}001437).
a first amplification primer comprising at least 10, preferably 15 and even more preferably 20 nucleotide motifs of nucleotide sequence SEQ ID No. 19 and a second amplification primer comprising at least 10, preferably 15 and even more preferably 20 nucleotide motifs of nucleotide sequence SEQ ID No. 20; indicatively, when the first primer has SEQ ID No. 19 as its sequence, and the second primer has SEQ ID No. 20 as its sequence, an amplicon is then obtained that is specific to the gene coding for HER2, with a size of 928 base pairs, which corresponds to sequence 2123-3051 on the reference sequence coding for HER2 (Genbank NM_{—}004448).

According to a particular embodiment of the invention, said pair of primers, used in stage B, comprises a first primer comprising a promoter permitting the initiation of transcription by a polymerase of bacteriophage T7, and is selected from the following pairs of primers:
a first amplification primer of SEQ ID No. 21 and a second amplification primer comprising at least 10, preferably 15 and even more preferably 20 nucleotide motifs of nucleotide sequence SEQ ID No. 2;
a first amplification primer of SEQ ID No. 22 and a second amplification primer comprising at least 10, preferably 15 and even more preferably 20 nucleotide motifs of nucleotide sequence SEQ ID No. 4;
a first amplification primer of SEQ ID No. 23 and a second amplification primer comprising at least 10, preferably 15 and even more preferably 20 nucleotide motifs of nucleotide sequence SEQ ID No. 6;
a first amplification primer of SEQ ID No. 24 and a second amplification primer comprising at least 10, preferably 15 and even more preferably 20 nucleotide motifs of nucleotide sequence SEQ ID No. 8.

In order to take account of the variability in enzymatic efficiency which may be observed in the various stages of the amplification reaction, the expression of a target gene can be standardized by simultaneous determination of the expression of a so-called housekeeping gene, expression of which is similar in different groups of patients. By maintaining a ratio of expression of the target gene to expression of the housekeeping gene, any variability between different experiments can be corrected. A person skilled in the art can refer notably to the following publications: Bustin S A Journal of molecular endocrinology, 2002, 29: 23-39; Giulietti A Methods, 2001, 25: 386-401. According to a particular embodiment of the invention, in stage B, at least one pair of amplification primers is used additionally, for obtaining amplicons specific to a housekeeping gene. By housekeeping gene, we mean a gene whose expression is stable in a given tissue, regardless of the physiological situation. According to a preferred embodiment of the invention, the housekeeping gene is the PPIB gene which codes for cyclophilin B. According to a preferred embodiment of the invention, said amplification primer for obtaining amplicons specific to a housekeeping gene comprises at least 10, preferably 15 and even more preferably 20 nucleotide motifs of a sequence selected from SEQ ID No. 25 to 29.

According to a particular embodiment of the invention, said pair of amplification primers for obtaining amplicons specific to a housekeeping gene is selected from the following pairs of primers:
a first amplification primer comprising at least 10, preferably 15 and even more preferably 20 nucleotide motifs of nucleotide sequence SEQ ID No. 27 and a second amplification primer comprising at least 10, preferably 15 and even more preferably 20 nucleotide motifs of nucleotide sequence SEQ ID No. 28; indicatively, when the first primer has SEQ ID No. 27 as its sequence, and the second primer has SEQ ID No. 28 as its sequence, an amplicon is obtained that is specific to the PPIB gene, with a size of 239 base pairs, which corresponds to sequence 231-470 on the PPIB reference sequence (Genbank M60857);
a first amplification primer comprising at least 10, preferably 15 and even more preferably 20 nucleotide motifs of nucleotide sequence SEQ ID No. 25 and a second amplification primer comprising at least 10, preferably 15 and even more preferably 20 nucleotide motifs of nucleotide sequence SEQ ID No. 26; indicatively, when the first primer has SEQ ID No. 25 as its sequence, and the second primer has SEQ ID No. 26 as its sequence, an amplicon is obtained that is specific to the PPIB gene, with a size of 639 base pairs, which corresponds to sequence 11-650 on the PPIB reference sequence (Genbank M60857).

According to a particular embodiment of the invention, said pair of amplification primers used for obtaining amplicons specific to a housekeeping gene comprises a first primer comprising a promoter permitting the initiation of transcription by a polymerase of bacteriophage T7. Said first amplification primer is preferably of SEQ ID No. 30 and said second amplification primer comprises preferably at least 10, preferably 15 and even more preferably 20 nucleotide motifs of nucleotide sequence SEQ ID No. 28.

In stage C, at least one detection probe is used for detecting the presence of said amplicons. This detection stage can be carried out by all the protocols known by a person skilled in the art relating to the detection of nucleic acids.

In the sense of the present invention, hybridization probe means a nucleic acid sequence of 10 to 100 nucleotide motifs, notably of 15 to 35 nucleotide motifs, possessing hybridization specificity in defined conditions for forming a hybridization complex with a target nucleic acid sequence. The hybridization probe can include a marker that permits it to be detected. They are then called detection probes. Detection means either direct detection by a physical method, or indirect detection by a method of detection using a marker. There are a great many methods of detection for the detection of nucleic acids. [See for example Kricka et al., Clinical Chemistry, 1999, No. 45(4), p. 453-458 or Keller G. H. et al., DNA Probes, 2nd Ed., Stockton Press, 1993, sections 5 and 6, p. 173-249]. Marker means a tracer capable of producing a signal that can be detected. A non-limiting list of these tracers comprises enzymes which produce a signal that can be detected for example by colorimetry, fluorescence or luminescence, such as horseradish peroxidase, alkaline phosphatase, beta galactosidase, glucose-6-phosphate dehydrogenase; chromophores such as fluorescent, luminescent or coloring compounds; groups with electron density detectable by electron microscopy or by their electrical properties such as conductivity, by the methods of amperometry or voltammetry, or by measurement of impedance; groups detectable by optical methods such as diffraction, surface plasmon resonance, variation of contact angle or by physical methods such as atomic force spectroscopy, the tunnel effect, etc.; radioactive molecules such as ³²P, ³⁵S or ¹²⁵I.

In the sense of the present invention, the hybridization probe can be a so-called detection probe. In this case, the so-called detection probe is labeled by means of a marker as defined previously. Owing to the presence of this marker, it is possible to detect the presence of a hybridization reaction between a given detection probe and the target sequence specific to a given species.

The detection probe can notably be a “molecular beacon” detection probe as described by Tyagi & Kramer (Nature biotech, 1996, 14: 303-308). These “molecular beacons” become fluorescent on hybridization. They possess a stem-loop structure and contain a fluorophore and a “quencher” group. Fixation of the specific loop sequence with its complementary sequence of target nucleic acid causes unwinding of the stem and emission of a fluorescent signal upon excitation at the appropriate wavelength.

The hybridization probe can also be a so-called capture probe. In this case, the so-called capture probe is or can be immobilized on a solid support by any appropriate means, i.e. directly or indirectly, for example by covalent bonding or by adsorption. The hybridization reaction between a given capture probe and a target sequence is then detected.

For the detection of the hybridization reaction, it is possible to use labeled target sequences, directly (notably by incorporating a marker within the target sequence) or indirectly (notably by the use of a detection probe as defined previously) the target sequence. Notably, before the hybridization stage it is possible to carry out a stage of labeling and/or cleavage of the target sequence, for example using a labeled deoxyribonucleotide triphosphate in the reaction of enzymatic amplification. Cleavage can be carried out notably by the action of imidazole and manganese chloride. The target sequence can also be labeled after the amplification stage, for example by hybridizing a detection probe according to the technique of sandwich hybridization described in document WO 91/19812. Another particular preferred manner of labeling nucleic acids is described in application FR2 780 059.

As solid support, it is possible to use synthetic materials or natural materials, optionally chemically modified, notably polysaccharides such as materials based on cellulose, for example paper, cellulose derivatives such as cellulose acetate and nitrocellulose or dextran, polymers, copolymers, notably based on monomers of the styrene type, natural fibres such as cotton, and synthetic fibres such as nylon; minerals such as silica, quartz, glasses, ceramics; latexes; magnetic particles; metallic derivatives, gels, etc. The solid support can be in the form of a microtitration plate, a membrane as described in application WO 94/12670, or a particle.

According to a preferred embodiment of the invention, the detection probe comprises a fluorophore and a quencher. According to an even more preferred embodiment of the invention, the hybridization probe comprises a fluorophore FAM (6-carboxy-fluorescein) or ROX (6-carboxy-X-rhodamine) at its 5′ end and a quencher (Dabsyl) at its 3′ end. Hereinafter, such a hybridization probe is called a “molecular beacon”.

According to a preferred embodiment of the invention, stages B and C are carried out simultaneously. This preferred embodiment can be employed in “real-time NASBA” which combines the technique of NASBA amplification and real-time detection using “molecular beacons” in a single stage. The NASBA reaction takes place in the tube, producing single-stranded RNA with which the specific “molecular beacons” can hybridize simultaneously to give a fluorescent signal. The formation of new RNA molecules is measured in real time by continuous monitoring of the signal in a fluorescent reader. In contrast to amplification by RT-PCR, amplification in NASBA can be carried out in the presence of DNA in the specimen. Therefore it is not necessary to verify that the DNA was in fact removed completely during extraction of the RNA.

As shown in the following example, when we wish to detect the target gene coding for ESR1 (reference sequence NCBI accession number: X03635), the following are preferably used in stage b):
a first primer of SEQ ID No. 1 or 21,
a second primer of SEQ ID No. 2
   and in stage c)
a detection probe comprising SEQ ID No. 9.

As shown in the following example, when we wish to detect the target gene coding for PGR (reference sequence NCBI accession number: NM_{—}000926), the following are preferably used in stage b):
a first primer of SEQ ID No. 3 or 22,
a second primer of SEQ ID No. 4
   and in stage c)
a detection probe comprising SEQ ID No. 10.

As shown in the following example, when we wish to detect the target gene coding for ESR2 (reference sequence NCBI accession number: MN_{—}001437), the following are preferably used in stage b):
a first primer of SEQ ID No. 5 or 23,
a second primer of SEQ ID No. 6
   and in stage c)
a detection probe comprising SEQ ID No. 11.

As shown in the following example, when we wish to detect the target gene coding for HER2 (reference sequence NCBI accession number: NM_{—}00448), the following are preferably used in stage b):
a first primer of SEQ ID No. 7 or 24,
a second primer of SEQ ID No. 8
   and in stage c)
a detection probe comprising SEQ ID No. 12.

As shown in the following example, when we wish to detect the PPIB housekeeping gene (reference sequence NCBI accession number: M60857), the following are preferably used in stage b):
a first primer of SEQ ID No. 27 or 30,
a second primer of SEQ ID No. 28
   and in stage c)
a detection probe comprising SEQ ID No. 29.

When using, in stage B, a pair of amplification primers for obtaining amplicons specific to a housekeeping gene, said amplicons specific to a housekeeping gene can be detected in a comparable manner to that described previously, notably by the use of a detection probe. According to a preferred embodiment of the invention, the housekeeping gene is the PPIB gene which codes for cyclophilin B and the detection probe comprises at least 10, preferably 15 and even more preferably 20 nucleotide motifs of a nucleotide sequence selected from SEQ ID No. 27 to 29. Preferably, this detection probe comprises a fluorophore and a quencher.

The invention also relates to an amplification primer comprising at least 10, preferably 15, and even more preferably 20 nucleotide motifs of a nucleotide sequence selected from SEQ ID No. 1 to SEQ ID No. 20.

According to a preferred embodiment of the invention, the amplification primer comprises a promoter permitting the initiation of transcription by a polymerase of bacteriophage T7. This primer can notably be any one of SEQ ID No. 21 to 24, and is preferably used in a NASBA amplification reaction.

The invention also relates to a pair of primers selected from the following pairs of primers:
a first amplification primer comprising at least 10, preferably 15 and even more preferably 20 nucleotide motifs of nucleotide sequence SEQ ID No. 1 and a second amplification primer comprising at least 10, preferably 15 and even more preferably 20 nucleotide motifs of nucleotide sequence SEQ ID No. 2; indicatively, when the first primer has SEQ ID No. 1 as its sequence, and the second primer has SEQ ID No. 2 as its sequence, an amplicon is obtained that is specific to the gene coding for ESR1, with a size of 202 base pairs, which corresponds to sequence 1427-1629 on the reference sequence coding for ESR1 (Genbank X03635).
a first amplification primer comprising at least 10, preferably 15 and even more preferably 20 nucleotide motifs of nucleotide sequence SEQ ID No. 3 and a second amplification primer comprising at least 10, preferably 15 and even more preferably 20 nucleotide motifs of nucleotide sequence SEQ ID No. 4; indicatively, when the first primer has SEQ ID No. 3 as its sequence, and the second primer has SEQ ID No. 4 as its sequence, an amplicon is then obtained that is specific to the gene coding for PGR, with a size of 184 base pairs, which corresponds to sequence 2761-2945 on the reference sequence coding for PGR (Genbank NM_{—}000926).
a first amplification primer comprising at least 10, preferably 15 and even more preferably 20 nucleotide motifs of nucleotide sequence SEQ ID No. 5 and a second amplification primer comprising at least 10, preferably 15 and even more preferably 20 nucleotide motifs of nucleotide sequence SEQ ID No. 6; indicatively, when the first primer has SEQ ID No. 5 as its sequence, and the second primer has SEQ ID No. 6 as its sequence, an amplicon is then obtained that is specific to the gene coding for ESR2, with a size of 210 base pairs, which corresponds to sequence 1640-1850 on the reference sequence coding for ESR2 (Genbank MN_{—}001437).
a first amplification primer comprising at least 10, preferably 15 and even more preferably 20 nucleotide motifs of nucleotide sequence SEQ ID No. 7 and a second amplification primer comprising at least 10, preferably 15 and even more preferably 20 nucleotide motifs of nucleotide sequence SEQ ID No. 8; indicatively, when the first primer has SEQ ID No. 7 as its sequence, and the second primer has SEQ ID No. 8 as its sequence, an amplicon is then obtained that is specific to the gene coding for HER2, with a size of 185 base pairs, which corresponds to sequence 2567-2752 on the reference sequence coding for HER2 (Genbank MN_{—}004448).
a first amplification primer comprising at least 10, preferably 15 and even more preferably 20 nucleotide motifs of nucleotide sequence SEQ ID No. 13 and a second amplification primer comprising at least 10, preferably 15 and even more preferably 20 nucleotide motifs of nucleotide sequence SEQ ID No. 14; indicatively, when the first primer has SEQ ID No. 13 as its sequence, and the second primer has SEQ ID No. 14 as its sequence, an amplicon is obtained that is specific to the gene coding for ESR1, with a size of 858 base pairs, which corresponds to sequence 808-1666 on the reference sequence coding for ESR1 (Genbank X03635).
a first amplification primer comprising at least 10, preferably 15 and even more preferably 20 nucleotide motifs of nucleotide sequence SEQ ID No. 15 and a second amplification primer comprising at least 10, preferably 15 and even more preferably 20 nucleotide motifs of nucleotide sequence SEQ ID No. 16; indicatively, when the first primer has SEQ ID No. 15 as its sequence, and the second primer has SEQ ID No. 16 as its sequence, an amplicon is then obtained that is specific to the gene coding for PGR, with a size of 658 base pairs, which corresponds to sequence 2319-2977 on the reference sequence coding for PGR (Genbank NM_{—}000926).
a first amplification primer comprising at least 10, preferably 15 and even more preferably 20 nucleotide motifs of nucleotide sequence SEQ ID No. 17 and a second amplification primer comprising at least 10, preferably 15 and even more preferably 20 nucleotide motifs of nucleotide sequence SEQ ID No. 18; indicatively, when the first primer has SEQ ID No. 17 as its sequence, and the second primer has SEQ ID No. 18 as its sequence, an amplicon is then obtained that is specific to the gene coding for ESR2, with a size of 702 base pairs, which corresponds to sequence 1246-1948 on the reference sequence coding for ESR2 (Genbank MN_{—}001437).
a first amplification primer comprising at least 10, preferably 15 and even more preferably 20 nucleotide motifs of nucleotide sequence SEQ ID No. 19 and a second amplification primer comprising at least 10, preferably 15 and even more preferably 20 nucleotide motifs of nucleotide sequence SEQ ID No. 20; indicatively, when the first primer has SEQ ID No. 19 as its sequence, and the second primer has SEQ ID No. 20 as its sequence, an amplicon is then obtained that is specific to the gene coding for HER2, with a size of 928 base pairs, which corresponds to sequence 2123-3051 on the reference sequence coding for HER2 (Genbank MN_{—}004448).

According to a preferred embodiment of the invention, said first primer comprises a promoter permitting the initiation of transcription by a polymerase of bacteriophage T7. This primer can notably be any one of SEQ ID 21 to 24. When the first primer comprises a promoter permitting the initiation of transcription by a polymerase of bacteriophage T7, this first primer is preferably included in a pair of primers selected from the following pairs of primers:
a first amplification primer of SEQ ID No. 21 and a second amplification primer comprising at least 10, preferably 15 and even more preferably 20 nucleotide motifs of nucleotide sequence SEQ ID No. 2;
a first amplification primer of SEQ ID No. 22 and a second amplification primer comprising at least 10, preferably 15 and even more preferably 20 nucleotide motifs of nucleotide sequence SEQ ID No. 4;
a first amplification primer of SEQ ID No. 23 and a second amplification primer comprising at least 10, preferably 15 and even more preferably 20 nucleotide motifs of nucleotide sequence SEQ ID No. 6;
a first amplification primer of SEQ ID No. 24 and a second amplification primer comprising at least 10, preferably 15 and even more preferably 20 nucleotide motifs of nucleotide sequence SEQ ID No. 8.

The invention also relates to the use of at least one amplification primer as defined previously and/or of at least one pair of primers as defined previously in a NASBA amplification reaction.

The invention also relates to an amplification primer for obtaining amplicons specific to a housekeeping gene. This amplification primer comprises preferably at least 10, preferably 15 and even more preferably 20 nucleotide motifs of a sequence selected from SEQ ID No. 25 to 29.

The invention also relates to a pair of amplification primers for obtaining amplicons specific to a housekeeping gene, selected from the following pairs:
a first amplification primer comprising at least 10, preferably 15 and even more preferably 20 nucleotide motifs of nucleotide sequence SEQ ID No. 27 and a second amplification primer comprising at least 10, preferably 15 and even more preferably 20 nucleotide motifs of nucleotide sequence SEQ ID No. 28; indicatively, when the first primer has SEQ ID No. 27 as its sequence, and the second primer has SEQ ID No. 28 as its sequence, an amplicon is obtained that is specific to the PPIB gene, with a size of 239 base pairs, which corresponds to sequence 231-470 on the reference sequence PPIB (Genbank M60857);

a first amplification primer comprising at least 10, preferably 15 and even more preferably 20 nucleotide motifs of nucleotide sequence SEQ ID No. 25 and a second amplification primer comprising at least 10, preferably 15 and even more preferably 20 nucleotide motifs of nucleotide sequence SEQ ID No. 26; indicatively, when the first primer has SEQ ID No. 25 as its sequence, and the second primer has SEQ ID No. 26 as its sequence, an amplicon is obtained that is specific to the PPIB gene, with a size of 639 base pairs, which corresponds to sequence 11-650 on the reference sequence PPIB (Genbank M60857).

According to a particular embodiment of the invention, said pair of amplification primers used for obtaining amplicons specific to a housekeeping gene comprises a first primer comprising a promoter permitting the initiation of transcription by a polymerase of bacteriophage T7. Said first amplification primer is preferably of SEQ ID No. 30 and said second amplification primer comprises preferably at least 10, preferably 15 and even more preferably 20 nucleotide motifs of nucleotide sequence SEQ ID No. 28.

The invention also relates to a detection probe comprising at least 10, preferably 15 and even more preferably 20 nucleotide motifs of a nucleotide sequence selected from SEQ ID No. 1 to SEQ ID No. 20.

Preferably, this detection probe comprises a fluorophore and a quencher.

The invention also relates to a hybridization probe for detecting amplicons specific to a housekeeping gene. Preferably, this detection probe comprises at least 10, preferably 15 and even more preferably 20 nucleotide motifs of a nucleotide sequence selected from SEQ ID No. 27 to 29. Preferably, this detection probe comprises a fluorophore and a quencher.

The invention also relates to the use of at least one primer as defined previously and/or at least one pair of primers as defined previously and/or at least one detection probe as defined previously for diagnosis/prognosis of breast cancer.

The invention finally relates to a kit for diagnosis/prognosis of breast cancer comprising at least one primer as defined previously and/or at least one pair of primers as defined previously and/or at least one detection probe as defined previously.

As shown in the following example, when we wish to detect the target gene coding for ESR1, the kit preferably comprises
a first primer of SEQ ID No. 1 or 21
a second primer of SEQ ID No. 2
a detection probe comprising SEQ ID No. 9.

As shown in the following example, when we wish to detect the target gene coding for PGR, the kit preferably comprises
a first primer of SEQ ID No. 3 or 22
a second primer of SEQ ID No. 4
a detection probe comprising SEQ ID No. 10.

As shown in the following example, when we wish to detect the target gene coding for ESR2 (reference sequence NCBI accession number: MN_{—}001437), the kit preferably comprises
a first primer of SEQ ID No. 5 or 23
a second primer of SEQ ID No. 6
a detection probe comprising SEQ ID No. 11.

As shown in the following example, when we wish to detect the target gene coding for HER2 (reference sequence NCBI accession number: NM_{—}00448), the kit preferably comprises
a first primer of SEQ ID No. 7 or 24
a second primer of SEQ ID No. 8
a detection probe comprising SEQ ID No. 12.

As shown in the following example, and according to a particular embodiment of the invention, the kit comprises in addition
a first primer of SEQ ID No. 27 or 30
a second primer of SEQ ID No. 28
a detection probe comprising SEQ ID No. 29.

The following figure is given by way of illustration and is not in any way limiting. It will facilitate understanding of the invention.

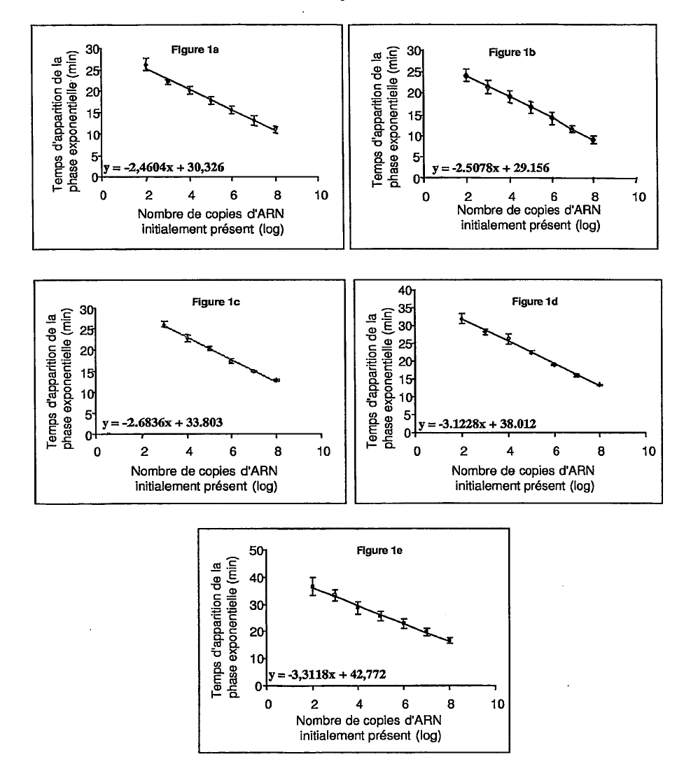

FIG. 1 shows the standard curves obtained for the genes ESR1 (FIG. 1a), PGR (FIG. 1b), ESR2 (FIG. 1c), HER2 (FIG. 1d), and PPIB (FIG. 1e) as described in example 1-3a. Each standard curve, obtained for each gene based on a reference sequence that is specific to the gene, transcribed to RNA in vitro in a plasmid, represents the time to appearance of the exponential phase of amplification (it is also called TTP: time to positivity, threshold time) as a function of the number of copies of RNA present at the start of NASBA amplification (the larger the initial number of RNA copies at the start of amplification, the shorter the time to appearance of the exponential phase).

The following examples are given by way of illustration and are not in any way limiting. They will facilitate understanding of the invention.

### EXAMPLE 1

### Amplification and Detection in Real Time of mRNAs Coding for ESR1, PGR, ESR2 and HER2

1/Obtaining and Preparing the Specimens

This example was carried out using three lines of tumor cells, whose expression of hormone receptors was previously determined by IHC or radioligand (or LBA), were used: MCF-7 (expressing the receptors ESR1 and PGR), T47D (not expressing the ESR1 receptor and expressing the PGR receptor) and BT-549 (expressing neither the ESR1 receptor, nor the PGR receptor). These lines were obtained from the American Type Culture Collection (ATCC, Manassas, USA). These cell lines were cultured in DMEM medium (MCF-7) or RPMI 1640 (T47D and BT-549), supplemented with fetal calf serum (10%), L-glutamine (2 mM), nonessential amino acids (1%) and streptomycin (10 μg/ml) at 37° C. under an atmosphere comprising 5% CO2.

This example was also carried out using tumors from patients (n=102) with breast cancer for which the expression of hormone receptors ER and PR (also called ESR1 and PGR) had previously been determined by radioligand (LBA) according to a conventional technique known by a person skilled in the art. The LBA technique informed us about the presence of functional receptors ER and PR in the cytosol of the cells. Expression of HER2 had previously been determined by quantitative PCR (providing us with information about amplification of the HER2 gene) and ELISA (providing information on expression of the membrane protein encoded by the HER2 gene) according to a conventional technique known by a person skilled in the art.

2/Extraction of Total RNAs

Total RNAs were extracted from cell lines using Trizol® Reagent according to the recommendations of the supplier of the kit (Invitrogen, Canada). The quality and quantity of RNA were determined at 260 and 280 nm and verified on agarose gel. The RNAs were then frozen at −70° C. until use.

Total RNAs were also extracted in a comparable manner from tumors from patients with breast cancer.

3/NASBA Amplificafion

The NASBA amplification reaction is based on the simultaneous activity of a reverse transcriptase of the avian myoblastosis virus (AMV-RT), RNase H of E. coli and RNA polymerase of bacteriophage T7 (Compton J, 1991, Nature, 350: 91-92). Real-time detection of the amplicons is carried out using a Nuclisens EasyQ® reader (bioMérieux BV, the Netherlands) and “molecular beacon” detection probes, as defined previously. Quantification in NASBA is based on the use of a standard curve, obtained starting from a reference sequence, specific to the target gene, transcribed to RNA in vitro in a plasmid. This standard curve represents the time to appearance of the exponential phase of amplification as a finction of the number of copies of RNA present at the start of NASBA amplification (the larger the initial number of RNA copies at the start of amplification, the shorter the time to appearance of the exponential phase).

a) Amplification of the Genes ESR1, PGR, ESR2, HER2 and of the PPIB Housekeeping Gene—Obtaining a Standard Curve

Standard Curve of the Target Gene Coding for ESR1

For the target gene coding for ESR1 (reference sequence NCBI accession number: X03635), a first primer of SEQ ID No. 13 5′ TACAGGCCAA ATTCAGATAA TCGAC 3′ and a second primer of SEQ ID No. 14 5′ GGAACCGAGAT GATGTAGCCA 3′ were used, located respectively in position 808-832 and 1646-1666 of the reference sequence, in order to generate by PCR (a first cycle of denaturation (95° C.; 1 min); then 35 cycles comprising the following stages: denaturation: 94° C.; 1 min; hybridization: 60° C.; 1 min; elongation: 72° C.; 2 min and a last cycle comprising a stage of denaturation: 72° C.; 7 min), an amplicon of 858 base pairs, specific to the gene coding for ESR1 (this will be called “ESR1 amplicon”).

The ESR1 amplicons obtained as described above were then cloned as plasmid pGEM-T (Promega, Madison, USA).

The sequence of these ESR1 amplicons was verified by sequencing (Biofidal, Vaulx en Velin, France), in order to ensure that it did indeed correspond to the sequence of the target gene that was to be amplified. The amplicons obtained were indeed specific to the ESR1 gene.

The amplicons were then transcribed to RNA in vitro using RNA polymerase (T7 or SP6 (Megascript® kit, Ambion, Austin, USA), depending on the orientation of the amplicon). After removing the plasmid by treatment with DNase, the RNAs were purified using the Rneasy® Mini Kit (Qiagen, Hilden, Germany) and quantified (RNA6000Nano, Agilent Technologies, Walbronn, Germany).

The RNAs obtained above were diluted to various concentrations (stock solution: 0.2×10¹¹ copies/μl, dilution in cascade 0.2×10¹¹ copies/μl to 0.2×10² copies/μl). These dilutions in cascade are amplified by NASBA using the Nuclisens basic® kit (bioMérieux BV, the Netherlands) in the presence of the specific primers ESR1 SEQ ID No. 1 and ESR1 No. 2 and of the “molecular beacons” SEQ ID No. 9:
0.2 μM of a first ESR1 primer of SEQ ID No. 1 5′ CTCCACCATG CCCTCTACAC A 3′, comprising, at its 5′ end, and shown in lower case, a sequence comprising the T7 polymerase promoter, i.e. a first primer whose complete sequence is SEQ ID No. 21: 5′ aattctaata cgactcacta tagggagaag gCTCCACCAT GCCCTCTACA CA 3′,
0.2 μM of a second ESR1 primer of SEQ ID No. 2 5′ ACATGATCAA CTGGGCGAAG A 3′,
0.1 μM of “molecular beacons” comprising SEQ ID No. 9 5′ GATCCTGATGATTGGTCTCG 3′, labeled with a fluorophore FAM (6-carboxyfluorescein) at 5′, and a “quencher” (Dabsyl) at 3′ (complete sequence: 5′ FAM-cgatcgGATC CTGATGATTG GTCTCGcgat cg-Dabsyl 3′).

As amplification proceeds, the signal intensifies in proportion to the quantity of amplicons produced. The curve of fluorescence as a function of time makes it possible to define the time at which the exponential phase of amplification will start (also called TTP: time to positivity, threshold time). The standard curve ESR1 connects the number of transcripts present initially in the solution as a function of the TTm detected in NASBA amplification. Using a standard curve, the number of copies of the target gene is calculated absolutely. Finally, this value is normalized on the basis of a housekeeping gene, in the present case the PPIB gene. This standard curve ESR1, shown FIG. 1a.

Standard Curve of the Target Gene Coding for PGR

The curve of the target gene coding for PGR was constructed according to the same principle as for ESR1, apart from the amplification primers used and the “molecular beacons”, which were specific to PGR.

Thus, for the target gene coding for PGR (reference sequence NCBI accession number: NM_{—}000926), a first primer of SEQ ID No. 15 5′ TGACAAGTCT TAATCAACTA GG 3′ and a second primer of SEQ ID No. 16 5′ TCACTTTTTAT GAAAGAGAAG GG 3′ were used, located respectively in position 2319-2340 and 2955-2977 of the reference sequence. The sequence of these PGR amplicons was verified by sequencing (Biofidal, Vaulx en Velin, France), in order to ensure that it did indeed correspond to the sequence of the target gene that was to be amplified. The amplicons obtained were indeed specific to the PGR gene.

The amplicons were then transcribed to RNA in vitro using RNA polymerase (T7 or SP6 (Megascript® kit, Ambion, Austin, USA), depending on the orientation of the amplicon). After removing the plasmid by treatment with DNase, the RNAs were purified using the Rneasy® Mini Kit (Qiagen, Hilden, Germany) and quantified (RNA6000Nano, Agilent Technologies, Walbronn, Germany).

The RNAs obtained above were diluted to various concentrations (stock solution: 0.2×10¹¹ copies/μl, dilution in cascade 0.2×10¹¹ copies/μl to 0.2×10² copies/μl). These dilutions in cascade are amplified by NASBA using the Nuclisens basic® kit (bioMérieux BV, the Netherlands) in the presence of:
0.1 μM of a first PGR primer of SEQ ID No. 3 5′ TCCCTGCCAA TATCTTGGGT A 3′, comprising, at its 5′ end, and shown in lower case, a sequence comprising the T7 polymerase promoter, i.e. a first primer whose complete sequence is SEQ ID No. 22: 5′ aattctaata cgactcacta tagggagaag gTCCCTGCCA ATATCTTGGG TA 3′,
0.1 μM of a second PGR primer of SEQ ID No. 4 5′ AGTTGTGTCG AGCTCACAGC 3′,
0.1 μM of “molecular beacons” used comprising SEQ ID No. 10 5′ CGGGCACTGAGTGTTGAATT 3′, labeled with a fluorophore FAM (6-carboxyfluorescein) at their 5′ end, and a “quencher” (Dabsyl) at its 3′ end (complete sequence: 5′ FAM-cgatcgCGGG CACTGAGTGT TGAATTcgat cg-Dabsyl 3′).

The standard curve PGR is shown in FIG. 1B.

Standard Curve of the Target Gene Coding for ESR2

The curve of the target gene coding for ESR2 was constructed according to the same principle as for ESR1, apart from the amplification primers used and the “molecular beacons”, which were specific to ESR2.

Thus, for the target gene coding for ESR2 (reference sequence NCBI accession number: MN_{—}001437), a first primer of SEQ ID No. 17 5′ GCCGCCCCAT GTGCTGAT 3′ and a second primer of SEQ ID No. 18 5′ GGACCCCGTGA TGGAGGACTT 3′ were used, located respectively in position 1246-1263 and 1928-1948 of the reference sequence. The sequence of these ESR2 amplicons was verified by sequencing (Biofidal, Vaulx en Velin, France), in order to ensure that it did indeed correspond to the sequence of the target gene that was to be amplified. The amplicons obtained were indeed specific to the ESR2 gene.

The amplicons were then transcribed to RNA in vitro using RNA polymerase (T7 or SP6 (Megascript® kit, Ambion, Austin, USA), depending on the orientation of the amplicon). After removing the plasmid by treatment with DNase, the RNAs were purified using the Rneasy® Mini Kit (Qiagen, Hilden, Germany) and quantified (RNA6000Nano, Agilent Technologies, Walbronn, Germany).

The RNAs obtained above were diluted to various concentrations (stock solution: 0.2×10¹¹ copies/μl, dilution in cascade 0.2×10¹¹ copies/μl to 0.2×10² copies/μl). These dilutions in cascade are amnplified by NASBA using the Nuclisens basic® kit (bioMérieux BV, the Netherlands) in the presence of:
0.2 μM of a first ESR2 primer of SEQ ID No. 5 5′ TGAGCAGATG TTCCATGCCC T 3′, comprising, at its 5′ end, and shown in lower case, the T7 polymerase promoter, i.e. a first primer whose complete sequence is SEQ ID No. 23: 5′ aattctaata cgactcacta tagggagaag gTGAGCAGAT GTTCCATGCC CT 3′,
0.2 μM of a second ESR2 primer of SEQ ID No. 6 5′ TCCAGTATGT ACCCTCTGGT 3′,
0.1 μM of “molecular beacons” comprising SEQ ID No. 11 5′ GATGCTTTGGTTTGGGTGAT 3′, labeled with a fluorophore FAM (6-carboxyfluorescein) at 5′, and a “quencher” (Dabsyl) at 3′.

The standard curve ESR2 is shown in FIG. 1C.

Standard Curve of the Target Gene Coding for HER2

The curve of the target gene coding for HER2 was constructed according to the same principle as for ESR1, apart from the amplification primers and the “molecular beacons”, which were specific to HER2.

Thus, for the target gene coding for HER2 (reference sequence NCBI accession number: NM_{—}00448), a first primer of SEQ ID No. 19 5′ TGGTTGGCAT TCTGCTGGTC GTGGT 3′ and a second primer of SEQ ID No. 20 5′ TGGCCGACAT TCAGAGTCAA TCATC 3′ were used, located respectively in position 2123-2147 and 3027-3051 of the reference sequence. The sequence of these HER2 amplicons was verified by sequencing (Biofidal, Vaulx en Velin, France), in order to ensure that it did indeed correspond to the sequence of the target gene that was to be amplified. The amplicons obtained were indeed specific to the HER2 gene.

The sequence of these HER2 amplicons was verified by sequencing (Biofidal, Vaulx en Velin, France), in order to ensure that it did indeed correspond to the sequence of the target gene that was to be amplified. The amplicons obtained were indeed specific to the HER2 gene.

The amplicons were then transcribed to RNA in vitro using RNA polymerase (T7 or SP6 (Megascript® kit, Ambion, Austin, USA), depending on the orientation of the amplicon). After removing the plasmid by treatment with DNase, the RNAs were purified using the Rneasy® Mini Kit (Qiagen, Hilden, Germany) and quantified (RNA6000Nano, Agilent Technologies, Walbronn, Germany).

The RNAs obtained above were diluted to various concentrations (stock solution: 0.2×10¹¹ copies/μl, dilution in cascade 0.2×10¹¹ copies/μl to 0.2×10² copies/μl). These dilutions in cascade are amplified by NASBA using the Nuclisens basic® kit (bioMérieux BV, the Netherlands) in the presence of:
0.2 μM of a first HER2 primer of SEQ ID No. 7 5′ GAGCCAGCCC GAAGTCTGTA 3′, comprising, at its 5′ end, and shown in lower case, the T7 polymerase promoter, i.e. a first primer whose complete sequence is SEQ ID No. 24: 5′ aattctaata cgactcacta tagggagaag g GAGCCAGC CCGAAGTCTG TA 3′,
0.2 μM of a second HER2 primer of SEQ ID No. 8 5′ TCTTAGACCA TGTCCGGGAA A 3′,
0.1 μM of “molecular beacons” used comprised SEQ ID No. 12 5′ GGAGGATGTG CGGCTCGTAC 3′, labeled with a fluorophore FAM (6-carboxyfluorescein) at their 5′ end, and a “quencher” (Dabsyl) at its 3′ end.
   The standard curve HER2 is shown in FIG. 1D.

Standard Curve of the PPIB Target Gene

The curve of the PPIB target gene was constructed according to the same principle as for ESR1, apart from the amplification primers and the “molecular beacons”, which were specific to PPIB.

Thus, for the PPIB housekeeping gene (reference sequence NCBI accession number: M60857), a first primer of SEQ ID No. 25 5′ ACATGAAGGT GCTCCTTGCC 3′ and a second primer of SEQ ID No. 26 5′ GTCCCTGTGC CCTACTCCTT 3′ were used, located respectively in position 11-30 and 631-650 of the reference sequence. The sequence of these PPIB amplicons was verified by sequencing (Biofidal, Vaulx en Velin, France), in order to ensure that it did indeed correspond to the sequence of the target gene that was to be amplified. The amplicons obtained were indeed specific to the PPIB gene.

The amplicons were then transcribed to RNA in vitro using RNA polymerase (T7 or SP6 (Megascript® kit, Ambion, Austin, USA), depending on the orientation of the amplicon). After removing the plasmid by treatment with DNase, the RNAs were purified using the Rneasy® Mini Kit (Qiagen, Hilden, Germany) and quantified (RNA6000Nano, Agilent Technologies, Walbronn, Germany).

The RNAs obtained above were diluted to various concentrations (stock solution: 0.2×10¹¹ copies/μl, dilution in cascade 0.2×10¹¹ copies/μl to 0.2×10² copies/μl). These dilutions in cascade are amplified by NASBA using the Nuclisens basic® kit (bioMérieux BV, the Netherlands) in the presence of:
0.2 μM of a first PPIB primer of SEQ ID No. 27 5′ CAGGCTGTCT TGACTGTCGT GA 3′, comprising, at its 5′ end, and shown in lower case, a sequence comprising the T7 polymerase promoter, i.e. a first primer whose complete sequence is SEQ ID No. 30: 5′ aattctaata cgactcacta tagggagaag gCAGGCTGTC TTGACTGTCG TGA 3′,
0.2 μM of a second PPIB primer of SEQ ID No. 28 5′ AGGAGAGAAA GGATTTGGCT 3′,
0.1 μM of “molecular beacons” comprising SEQ ID No. 29 5′ GATCCAGGGCGGAGACTTCA 3′, labeled with a fluorophore ROX (6-carboxy-X-rhodamine) at 5′, and a “quencher” (Dabsyl) at 3′ (complete sequence: 5′ ROX-cgatcgGATC CAGGGCGGAG ACTTCAcgat cg-Dabsyl 3′).

The standard curve PPIB is shown in FIG. 1E.

b) Reaction of NASBA Amplification:

b1) Amplification in Duplex of the ESR1 and PPIB Genes

This amplification reaction was carried out using a Nuclisens basic® kit (bioMérieux BV, the Netherlands). For this, 5 ng of total RNA extracted from different cell lines was added to 10 μl of NASBA buffer (final concentration in 20 μl of reaction medium: 40 mM of Tris HCl pH 8.5, 12 mM MgCl2, 70 mM KCl, 5 mM dithiothreitol, 15% v/v DMSO, 1 mM of each dNTP, 2 mM of each NTP).

0.1 μM of “molecular beacons” comprising
SEQ ID No. 9 5′ GATCCTGATGATTGGTCTCG 3′, labeled with a fluorophore FAM (6-carboxyfluorescein) at 5′, and a “quencher” (Dabsyl) at 3′ (complete sequence: 5′ FAM-cgatcgGATC CTGATGATTG GTCTCGcgat cg-Dabsyl 3′, for detecting the RNAs of the ESR1 gene),
SEQ ID No. 29 5′ GATCCAGGGCGGAGACTTCA 3′, labeled with a fluorophore ROX (6-carboxy-X-rhodamine) at 5′, and a “quencher” (Dabsyl) at 3′ (complete sequence: 5′ ROX-cgatcgGATC CAGGGCGGAG ACTTCAcgat cg-Dabsyl 3′, for detecting the RNAs of the PPIB gene),
   was added to this medium.

The following were also added to this medium:
0.2 μM of a first ESR1 primer of SEQ ID No. 1 5′ CTCCACCATG CCCTCTACAC A 3′, comprising, at its 5′ end, and shown in lower case, a sequence comprising the T7 polymerase promoter, i.e. a first primer whose complete sequence is SEQ ID No. 21: 5′ aattctaata cgactcacta tagggagaag gCTCCACCAT GCCCTCTACA CA 3′,
0.2 μM of a second ESR1 primer of SEQ ID No. 2 5′ ACATGATCAA CTGGGCGAAG A 3′,
0.2 μM of a first PPIB primer of SEQ ID No. 27 5′ CAGGCTGTCT TGACTGTCGT GA 3′, comprising, at its 5′ end, and shown in lower case, a sequence comprising the T7 polymerase promoter, i.e. a first primer whose complete sequence is SEQ ID No. 30: 5′ aattctaata cgactcacta tagggagaag gCAGGCTGTC TTGACTGTCG TGA 3′,
0.2 μM of a second PPIB primer of SEQ ID No. 28 5′ AGGAGAGAAA GGATTTGGCT 3′.

Preincubation was carried out for 2 minutes at 65° C. prior to incubation of 2 minutes at 41° C. A volume of 5 μl of an enzyme mixture (0.08 U of RNase H, 32 U of RNA polymerase T7, 6.4 U of AMV-RT) was added, and incubation of 90 minutes was carried out at 41° C.

The transcribed RNAs were quantified in real time (NucliSens EasyQ, bioMérieux), the NASBA reaction producing amplicons with which the specific “molecular beacons” can hybridize simultaneously to give a fluorescent signal. Formation of the new RNA molecules is measured in real time by continuous monitoring of the signal in a fluorescent reader, the NucliSens EasyQ analyzer. Analysis and automated reporting of the results are provided by Nuclisens TTP software (bioMérieux BV, the Netherlands). The standard curve, as defined previously (transcribed RNA dilution: 10⁸ to 10² copies), was used for quantifying the expression of each target ESR1 gene and of the PPIB housekeeping gene, in order to extrapolate the number of mRNA copies per specimen. The quantification of the expression of a target gene was expressed as the number of mRNA copies/5 ng of total RNA.

Table 1 shows the expression of the ESR1 gene quantified using 5 ng of total RNA derived from cell lines MCF-7, T47D and BT-549.

TABLE 1
Expression of the ESR1 gene in MCF-7, T47D,
BT-549 cells (NC: cannot be calculated)

| | Number of mRNA | Number of mRNA | |
|---|---|---|---|
| | copies ESR1 | copies PPIB | ESR1/PPIB |
| MCF-7 | 2.24 × 10⁴ | 7.43 × 10⁵ | 3.01 × 10⁻² |
| T47D | 3.24 × 10⁴ | 2.34 × 10⁶ | 1.38 × 10⁻² |
| BT549 | NC | 4.43 × 10⁶ | NC |

The expression of the ESR1 gene was expressed as the ratio of the number of mRNA copies of the target gene to the number of mRNA copies of the housekeeping gene. Thus, mRNAs of ESR1 were expressed in the MCF-7 cells whereas they were not detected in the BT-549 cells, in agreement with the expression in hormone receptors of these cells. Note that mRNAs of ESR1 were expressed in the T47D cells, whereas only the PGR receptor was expressed, suggesting post-transcriptional regulation of the ESR1 gene.

Table 2 shows the expression of the ESR1 gene quantified using 50 ng of total RNA obtained from IHC+ tumors, i.e. expressing nuclear hormone receptors of the tumor cells, or from IHC− tumors, i.e. not expressing nuclear hormone receptors (average of 3-7 tumors).

TABLE 2
Expression of the ESR1 gene in IHC+ and IHC− tumors

| | Average mRNA | Average mRNA | |
|---|---|---|---|
| | copies ESR1 | copies PPIB | ESR1/PPIB |
| IHC+ tumors | 2.64 × 10⁵ | 4.71 × 10⁶ | 5.61 × 10⁻² |
| IHC− tumors | 9.62 × 10³ | 9.39 × 10⁶ | 1.02 × 10⁻³ |

The expression of the ESR1 gene was expressed as the ratio of the number of mRNA copies of the target gene to the number of mRNA copies of the housekeeping gene. Overexpression of the ESR1 gene was observed in the IHC+ tumors.

On the basis of the ESR1 and PPIB standard curves constructed previously, the limit of detection and the limit of quantification were determined for the ESR1 and PPIB genes amplified in duplex. The limit of quantification was observed at 100 mRNA copies for ESR1 and PPIB. The limit of detection was 100 mRNA copies for ESR1 and PPIB.

The specificity of the ESR1/PPIB duplex was tested by amplifying ESR1 in the presence of a “molecular beacon” specific to PGR, and by amplifying PGR in the presence of a “molecular beacon” specific to ESR1. No signal was detected. Similarly, no signal was observed when NASBA was performed starting with total RNAs obtained from the cell line BT-549, ESR1 and PGR negative.

All these results were confirmed using another amplification technique (quantitative RT-PCR). Moreover, the results obtained at the messenger RNA level by the NASBA technique for the ESR1 gene were correlated with those obtained at the protein level by LBA (Ligand Binding Assay) (ESR: r=0.77, p<0.0001; Spearman statistical test of correlation). These results demonstrate that the expression of mRNAs of ESR1 is correlated with the presence of the functional receptor in the cytosol, confirming the benefit of investigating the expression of this gene at the mRNA level.

These results demonstrate that the ESR1/PPIB duplex in NASBA permits the expression of the ESR1 gene to be quantified on the basis of a very small quantity of total RNA, and, more broadly, on the basis of a very small quantity of tumor cells, and is entirely suitable for investigating the diagnosis/prognosis of breast cancer, and a patient's response to a particular treatment.

b2) Amplification in Duplex of the PGR and PPIB Genes

This amplification reaction was carried out using a Nuclisens basic® kit (bioMérieux BV, the Netherlands). For this, 5 ng of total RNA extracted from different cell lines was added to 10 μl of NASBA buffer (final concentration in 20 μl of reaction medium: 40 mM of Tris HCl pH 8.5, 12 mM MgCl2, 70 mM KCl, 5 mM dithiothreitol, 15% v/v DMSO, 1 mM of each dNTP, 2 mM of each NTP).

0.1 μM of “molecular beacons” comprising
SEQ ID No. 10 5′ CGGGCACTGAGTGTTGAATT 3′, labeled with a fluorophore FAM (6-carboxyfluorescein) at their 5′ end, and a “quencher” (Dabsyl) at its 3′ end (complete sequence: 5′ FAM-cgatcgCGGG CACTGAGTG T TGAATTcg at cg-Dabsyl 3′) for detecting the RNAs coding for PGR during the PGR/cyclophilin B duplex,
SEQ ID No. 29 5′ GATCCAGGGCGGAGACTTCA 3′, labeled with a fluorophore ROX (6-carboxy-X-rhodamine) at 5′, and a “quencher” (Dabsyl) at 3′ (complete sequence: 5′ ROX-cgatcgGATC CAGGGCGGAG ACTTCAcgat cg-Dabsyl 3′, for detecting the RNAs of the PPIB gene),
   was added to this medium.

The following were also added to this medium:
0.1 μM of a first PGR primer of SEQ ID No. 3 5′ TCCCTGCCAA TATCTTGGGT A 3′, comprising, at its 5′ end, and shown in lower case, a sequence comprising the T7 polymerase promoter, i.e. a first primer whose complete sequence is SEQ ID No. 22: 5′ aattctaata cgactcacta tagggagaag gTCCCTGCCA ATATCTTGGG TA 3′,
0.1 μM of a second PGR primer of SEQ ID No. 4 5′ AGTTGTGTCG AGCTCACAGC 3′,
0.2 μM of a first PPIB primer of SEQ ID No. 27 5′ CAGGCTGTCT TGACTGTCGT GA 3′, comprising, at its 5′ end, and shown in lower case, a sequence comprising the T7 polymerase promoter, i.e. a first primer whose complete sequence is SEQ ID No. 30: 5′ aattctaata cgactcacta tagggagaag gCAGGCTGTC TTGACTGTCG TGA 3′,
0.2 μM of a second PPIB primer of SEQ ID No. 28 5′ AGGAGAGAAA GGATTTGGCT 3′.

Preincubation was carried out for 2 minutes at 65° C. prior to incubation of 2 minutes at 41° C. A volume of 5 μl of an enzyme mixture (0.08 U of RNase H, 32 U of RNA polymerase T7, 6.4 U of AMV-RT) was added, and incubation of 90 minutes was carried out at 41° C.

The transcribed RNAs were quantified in real time according to a principle comparable to that described for ESR1. The standard curve, as defined previously (transcribed RNA dilution: 10⁸ to 10² copies) was used for quantifying the expression of the target PGR gene and of the PPIB housekeeping gene, in order to extrapolate the number of mRNA copies per specimen. The quantification of the expression of a target gene was expressed as the number of mRNA copies/5 ng of total RNA.

Table 3 shows the expression of the PGR gene quantified using 5 ng of total RNA derived from cell lines MCF-7, T47D and BT-549.

TABLE 3
Expression of the PGR gene in MCF-7, T47D, BT-549 cells

| | Number of mRNA | Number of mRNA | |
|---|---|---|---|
| | copies PGR | copies PPIB | PGR/PPIB |
| MCF-7 | 4.35 × 10² | 2.54 × 10⁶ | 1.71 × 10⁻⁴ |
| T47D | 3.92 × 10⁴ | 8.32 × 10⁵ | 4.71 × 10⁻² |
| BT549 | NC | 9.73 × 10⁶ | NC |

The expression of the PGR gene was expressed as the ratio of the number of mRNA copies of the target gene to the number of mRNA copies of the housekeeping gene. Thus, mRNAs of PGR were expressed in the MCF-7 and T47D cells whereas they were not detected in the BT-549 cells, in agreement with the expression of hormone receptors of these cells.

Table 4 shows the expression of the PGR gene quantified using 50 ng of total RNA obtained from IHC+ tumors, i.e. expressing hormone receptors on the surface of the tumor cells, or from IHC− tumors, i.e. not expressing hormone receptors on their surface (average of 3-7 tumors).

TABLE 4
Expression of the PGR gene in IHC+ and IHC− tumors

| | Average mRNA | Average mRNA | |
|---|---|---|---|
| | copies PGR | copies PPIB | PGR/PPIB |
| IHC+ tumors | 2.78 × 10³ | 9.23 × 10⁷ | 3.01 × 10⁻⁴ |
| IHC− tumors | 2.98 × 10³ | 1.91 × 10⁶ | 1.56 × 10⁻⁴ |

The expression of the PGR gene was expressed as the ratio of the number of mRNA copies of the target gene to the number of mRNA copies of the housekeeping gene. Overexpression of the PGR gene was observed in the IHC+ tumors.

On the basis of the PGR and PPIB standard curves constructed previously, the limit of detection and the limit of quantification were determined for the PGR and PPIB genes amplified in duplex. The limit of quantification was determined at 100 and 1000 mRNA copies for PGR and PPIB respectively. The limit of detection was 100 mRNA copies for PGR and PPIB.

The specificity of the PGR/PPIB duplex was tested by amplifying PGR in the presence of a “molecular beacon” specific to ESR1. No signal was detected. Similarly, no signal was observed when NASBA was carried out using total RNAs obtained from the cell line BT-549, PGR negative.

All these results were confirmed using another amplification technique (quantitative RT-PCR). Moreover, the results obtained at the messenger RNA level by the NASBA technique for the PGR gene were correlated with those obtained at the protein level by LBA (Ligand Binding Assay) (PGR: r=0.87, p<0.0001, n=102; Spearman statistical test of correlation). These results demonstrate that the expression of mRNAs of PGR is correlated with the presence of the functional receptor in the cytosol, confirming the benefit of investigating the expression of this gene at the mRNA level.

These results demonstrate that the PGRIPPIB duplex in NASBA permits the expression of the PGR gene to be quantified on the basis of a very small quantity of total RNA, and, more broadly, on the basis of a very small quantity of tumor cells, and is entirely suitable for investigating the diagnosis/prognosis of breast cancer, and a patient's response to a particular treatment.

b3) Amplification in Duplex of the ESR2 and PPIB Genes

This amplification reaction was carried out using a Nuclisens basic® kit (bioMérieux BV, the Netherlands). For this, 5 ng of total RNA extracted from different cell lines was added to 10 μl of NASBA buffer (final concentration in 20 μl of reaction medium: 40 mM of Tris HCl pH 8.5, 12 mM MgCl2, 70 mM KCI, 5 mM dithiothreitol, 15% v/v DMSO, 1 mM of each dNTP, 2 mM of each NTP).

0.1 μM of “molecular beacons” comprising
SEQ ID No. 11 5′ GATGCTTTGGTTTGGGTGAT 3′, labeled with a fluorophore FAM (6-carboxyfluorescein) at 5′, and a “quencher” (Dabsyl) at 3′,

SEQ ID No. 29 5′ GATCCAGGGCGGAGACTTCA 3′, labeled with a fluorophore ROX (6-carboxy-X-rhodamine) at 5′, and a “quencher” (Dabsyl) at 3′ (complete sequence: 5′ ROX-cgatcgGATC CAGGGCGGAG ACTTCAcgat cg-Dabsyl 3′, for detecting the RNAs of the PPIB gene coding for cyclophilin B),

was added to this medium.

The following were also added to this medium:
0.2 μM of a first ESR2 primer of SEQ ID No. 5 5′ TGAGCAGATG TTCCATGCCC T 3′, comprising, at its 5′ end, and shown in lower case, the T7 polymerase promoter, i.e. a first primer whose complete sequence is SEQ ID No. 23: 5′ aattctaata cgactcacta tagggagaag gTGAGCAGAT GTTCCATGCC CT 3′,
0.2 μM of a second ESR2 primer of SEQ ID No. 6 5′ TCCAGTATGT ACCCTCTGGT 3′,
0.2 μM of a first PPIB primer of SEQ ID No. 27 5′ CAGGCTGTCT TGACTGTCGT GA 3′, comprising, at its 5′ end, and shown in lower case, the T7 polymerase promoter, i.e. a first primer whose complete sequence is SEQ ID No. 30: 5′ aattctaata cgactcacta tagggagaag gCAGGCTGTC TTGACTGTCG TGA 3′, 0.2 μM of a second PPIB primer of SEQ ID No. 28 5′ AGGAGAGAAA GGATTTGGCT 3′.

Preincubation was carried out for 2 minutes at 65° C. prior to incubation of 2 minutes at 41° C. A volume of 5 μl of an enzyme mixture (0.08 U of RNase H, 32 U of RNA polymerase T7, 6.4 U of AMV-RT) was added, and incubation of 90 minutes was carried out at 41° C.

The transcribed RNAs were quantified in real time (NucliSens EasyQ, bioMérieux), the NASBA reaction producing amplicons with which the specific “molecular beacons” can hybridize simultaneously to give a fluorescent signal. The formation of the new RNA molecules is measured in real time by continuous monitoring of the signal in a fluorescent reader, the NucliSens EasyQ analyzer. Analysis and automated reporting of the results are provided by Nuclisens TTP software (bioMérieux BV, the Netherlands). The standard curve, as defined previously (transcribed RNA dilution: 10⁸ to 10² copies), was used for quantifying the expression of each target ESR2 gene and PPIB housekeeping gene, which made it possible to extrapolate the number of mRNA copies per specimen.

On the basis of the ESR2 and PPIB standard curves constructed previously, the limit of detection and the limit of quantification were determined for each of the genes. The limit of quantification was observed at 1000 and 10 000 mRNA copies for ESR2 and PPIB. The limit of detection was 1000 mRNA copies for ESR2 and PPIB.

The specificity of the ESR2/PPIB duplex was tested by amplifying ESR2 in the presence of a “molecular beacon” specific to HER2, and by amplifying HER2 in the presence of a “molecular beacon” specific to ESR2. No signal was detected.

These results demonstrate that the ESR2/PPIB duplex in NASBA permits specific and sensitive amplification of the ESR2 gene on the basis of a very small quantity of total RNA, making this duplex entirely suitable for investigating the diagnosis/prognosis of breast cancer, and a patient's response to a particular treatment.

b2) Amplification in Duplex of the HER2 and PPIB Genes

This amplification reaction was carried out using a Nuclisens basic® kit (bioMérieux BV, the Netherlands). For this, 5 ng of total RNA extracted from different cell lines was added to 10 μl of NASBA buffer (final concentration in 20 μl of reaction medium: 40 mM of Tris HCl pH 8.5, 12 mM MgCl2, 70 mM KCl, 5 mM dithiothreitol, 15% v/v DMSO, 1 mM of each dNTP, 2 mM of each NTP).

0.1 μM of “molecular beacons” comprising
SEQ ID No. 12 5′ GGAGGATGTG CGGCTCGTAC 3′, labeled with a fluorophore FAM (6-carboxyfluorescein) at their 5′ end, and a “quencher” (Dabsyl) at its 3′ end for detecting the RNAs coding for HER2 in the HER2/PPIB duplex,
SEQ ID No. 29 5′ GATCCAGGGC GGAGACTTCA 3′, labeled with a fluorophore ROX (6-carboxy-X-rhodamine) at 5′, and a “quencher” (Dabsyl) at 3′ (complete sequence: 5′ ROX-cgatcgGATC CAGGGCGGAG ACTTCAcgat cg-Dabsyl 3′, for detecting the RNAs of the PPIB gene coding for cyclophilin B,
   was added to this medium.

The following were also added to this medium:
0.2 μM of a first HER2 primer of SEQ ID No. 7 5′ GAGCCAGCCC GAAGTCTGTA 3′, comprising, at its 5′ end, and shown in lower case, the T7 polymerase promoter, i.e. a first primer whose complete sequence is SEQ ID No. 24: 5′ aattctaata cgactcacta tagggagaag g GAGCCAGC CCGAAGTCTG TA 3′,
0.2 μM of a second HER2 primer of SEQ ID No. 8 5′ TCTTAGACCA TGTCCGGGAA A 3′,
0.2 μM of a first cyclophilin B primer of SEQ ID No. 27 5′ CAGGCTGTCT TGACTGTCGT GA 3′, comprising, at its 5′ end, and shown in lower case, the T7 polymerase promoter, i.e. a first primer whose complete sequence is SEQ ID No. 30: 5′ aattctaata cgactcacta tagggagaag gCAGGCTGTC TTGACTGTCG TGA 3′, 0.2 μM of a second cyclophilin B primer of SEQ ID No. 28 5′ AGGAGAGAAA GGATTTGGCT 3′.

Preincubation was carried out for 2 minutes at 65° C. prior to incubation of 2 minutes at 41° C. A volume of 5 μl of an enzyme mixture (0.08 U of RNase H, 32 U of RNA polymerase T7, 6.4 U of AMV-RT) was added, and incubation of 90 minutes was carried out at 41° C.

The transcribed RNAs were quantified in real time (NucliSens EasyQ, bioMérieux), the NASBA reaction producing amplicons with which the specific “molecular beacons” can hybridize simultaneously to give a fluorescent signal. Formation of the new RNA molecules is measured in real time by continuous monitoring of the signal in a fluorescent reader, the NucliSens EasyQ analyzer. Analysis and automated reporting of the results are provided by Nuclisens TTP software (bioMérieux BV, the Netherlands). The standard curve, as defined previously (transcribed RNA dilution: 10⁸ to 10² copies) was used for quantifying the expression of each target HER2 gene and PPIB housekeeping gene, which made it possible to extrapolate the number of mRNA copies per specimen.

On the basis of the HER2 and PPIB standard curves constructed previously, the limit of detection and the limit of quantification were determined for each of the genes. The limit of quantification was determined at 1000 and 10 000 mRNA copies for HER2 and PPIB respectively. The limit of detection was 100 mRNA copies for HER2 and PPIB. The specificity of the HER2/PPIB duplex was tested by amplifying ESR2 in the presence of a “molecular beacon” specific to HER2, and by amplifying HER2 in the presence of a “molecular beacon” specific to ESR2. No signal was detected.

The results obtained at the messenger RNA level by the NASBA technique for the HER2 gene were correlated with the results obtained in quantitative PCR (r=0.54, p<0.0001, n=97, Spearman statistical test of correlation) and in ELISA (r=0.50, p<0.0001, n=93; Spearman statistical test of correlation). These results demonstrate that the expression of mRNAs of HER2 is correlated with the amplification of the gene as well as with overexpression of the HER2 membrane receptor, confining the benefit of investigating the expression of this gene at the mRNA level.

These results demonstrate that the HER2/PPIB duplex in NASBA permits specific and sensitive amplification of the HER2 gene on the basis of a very small quantity of total RNA, making this duplex entirely suitable for investigating the diagnosis/prognosis of breast cancer, and a patient's response to a particular treatment.

1. A method for diagnosis/prognosis of breast cancer comprising the following stages:
A—the nuclear material is extracted from a biological specimen,
B—at least one pair of amplification primers is used for obtaining amplicons of at least one target sequence of the nuclear material
C—at least one detection probe is used for detecting the presence of said amplicons
characterized in that, in stage B, said pair of primers comprises at least one amplification primer comprising at least 10 nucleotide motifs of a nucleotide sequence selected from SEQ ID No. 1 to SEQ ID No. 20 and/or in stage C, said detection probe comprises at least 10 nucleotide motifs of a nucleotide sequence selected from SEQ ID No. 1 to SEQ ID No. 20.

2. The method for diagnosis/prognosis of breast cancer as claimed in claim 1, characterized in that, in stage B, said pair of primers is selected from the following pairs of primers:
a first amplification primer comprising at least 10 nucleotide motifs of nucleotide sequence SEQ ID No. 1 and a second amplification primer comprising at least 10 nucleotide motifs of nucleotide sequence SEQ ID No. 2;
a first amplification primer comprising at least 10 nucleotide motifs of nucleotide sequence SEQ ID No. 3 and a second amplification primer comprising at least 10 nucleotide motifs of nucleotide sequence SEQ ID No. 4;
a first amplification primer comprising at least 10 nucleotide motifs of nucleotide sequence SEQ ID No. 5 and a second amplification primer comprising at least 10 nucleotide motifs of nucleotide sequence SEQ ID No. 6;
a first amplification primer comprising at least 10 nucleotide motifs of nucleotide sequence SEQ ID No. 7 and a second amplification primer comprising at least 10 nucleotide motifs of nucleotide sequence SEQ ID No. 8;
a first amplification primer comprising at least 10 nucleotide motifs of nucleotide sequence SEQ ID No. 13 and a second amplification primer comprising at least 10 nucleotide motifs of nucleotide sequence SEQ ID No. 14;
a first amplification primer comprising at least 10 nucleotide motifs of nucleotide sequence SEQ ID No. 15 and a second amplification primer comprising at least 10 nucleotide motifs of nucleotide sequence SEQ ID No. 16;
a first amplification primer comprising at least 10 nucleotide motifs of nucleotide sequence SEQ ID No. 17 and a second amplification primer comprising at least 10 nucleotide motifs of nucleotide sequence SEQ ID No. 18;
a first amplification primer comprising at least 10 nucleotide motifs of nucleotide sequence SEQ ID No. 19 and a second amplification primer comprising at least 10 nucleotide motifs of nucleotide sequence SEQ ID No. 20.

3. The method for diagnosis/prognosis of breast cancer as claimed in claim 1 in which said pair of primers comprises at least one amplification primer comprising a promoter permitting the initiation of transcription by a polymerase of bacteriophage T7.

4. The method for diagnosis/prognosis of breast cancer as claimed in claim 1 in which, in stage C, the detection probe comprises a fluorophore and a quencher.

5. The method as claimed in claim 1 in which the target sequence comprises a gene selected from ESR1, ESR2, PGR, HER2.

6. The method as claimed in claim 1 in which stages B and C are carried out simultaneously.

7. The method as claimed claim 1, characterized in that, in stage B, at least one pair of amplification primers is used additionally, for obtaining amplicons specific to a housekeeping gene.

8. The method as claimed in claim 7, characterized in that the amplification primer for obtaining amplicons specific to a housekeeping gene comprises at least 10 nucleotide motifs of a sequence selected from SEQ ID No. 25 to 29.

9. The method as claimed in claim 7, characterized in that said pair of amplification primers for obtaining amplicons specific to a housekeeping gene is selected from the following pairs of primers:
a first amplification primer comprising at least 10 nucleotide motifs of nucleotide sequence SEQ ID No. 27 and a second amplification primer comprising at least 10 nucleotide motifs of nucleotide sequence SEQ ID No. 28;
a first amplification primer comprising at least 10 nucleotide motifs of nucleotide sequence SEQ ID No. 25 and a second amplification primer comprising at least 10 nucleotide motifs of nucleotide sequence SEQ ID No. 26.

10. An amplification primer comprising at least 10 nucleotide motifs of a nucleotide sequence selected from SEQ ID No. 1 to SEQ ID No. 20; 25 to 29.

11. The amplification primer as claimed in claim 10, comprising a promoter permitting the initiation of transcription by a polymerase of bacteriophage T7.

12. A pair of amplification primers selected from the following pairs of primers:
a first amplification primer comprising at least 10 nucleotide motifs of nucleotide sequence SEQ ID No. 1 and a second amplification primer comprising at least 10 nucleotide motifs of nucleotide sequence SEQ ID No. 2;
a first amplification primer comprising at least 10 nucleotide motifs of nucleotide sequence SEQ ID No. 3 and a second amplification primer comprising at least 10 nucleotide motifs of nucleotide sequence SEQ ID No. 4;
a first amplification primer comprising at least 10 nucleotide motifs of nucleotide sequence SEQ ID No. 5 and a second amplification primer comprising at least 10 nucleotide motifs of nucleotide sequence SEQ ID No. 6;
a first amplification primer comprising at least 10 nucleotide motifs of nucleotide sequence SEQ ID No. 7 and a second amplification primer comprising at least 10 nucleotide motifs of nucleotide sequence SEQ ID No. 8;
a first amplification primer comprising at least 10 nucleotide motifs of nucleotide sequence SEQ ID No. 13 and a second amplification primer comprising at least 10 nucleotide motifs of nucleotide sequence SEQ ID No. 14;
a first amplification primer comprising at least 10 nucleotide motifs of nucleotide sequence SEQ ID No. 15 and a second amplification primer comprising at least 10 nucleotide motifs of nucleotide sequence SEQ ID No. 16;
a first amplification primer comprising at least 10 nucleotide motifs of nucleotide sequence SEQ ID No. 17 and a second amplification primer comprising at least 10 nucleotide motifs of nucleotide sequence SEQ ID No. 18;
a first amplification primer comprising at least 10 nucleotide motifs of nucleotide sequence SEQ ID No. 19 and a second amplification primer comprising at least 10 nucleotide motifs of nucleotide sequence SEQ ID No. 20.

13. The pair of primers as claimed in claim 12, in which said first primer comprises a promoter permitting the initiation of transcription by a polymerase of bacteriophage T7.

14. An amplification method comprising using at least one amplification primer as claimed in claim 10 in a NASBA amplification reaction.

15. A detection probe comprising at least 10 nucleotide motifs of a nucleotide sequence selected from SEQ ID No. 1 to SEQ ID No. 20.

16. The detection probe as claimed in claim 15, comprising a fluorophore and a quencher.

17. A method for diagnosis/prognosis of breast cancer comprising using for said diagnosis/prognosis at least one primer as claimed in claim 10.

18. A kit for diagnosis/prognosis of breast cancer comprising at least one primer as claimed in claim 10.

19. An amplification method comprising using at least one amplification primer as claimed in claim 11 in a NASBA amplification reaction.

20. An amplification method comprising using a pair of primers as claimed in claim 12 in a NASBA amplification reaction.

21. An amplification method comprising using a pair of primers as claimed in claim 13 in a NASBA amplification reaction.

22. A method for diagnosis/prognosis of breast cancer comprising using for said diagnosis/prognosis at least one primer as claimed in claim 11.

23. A method for diagnosis/prognosis of breast cancer comprising using for said diagnosis/prognosis at least one pair of primers as claimed in claim 12.

24. A method for diagnosis/prognosis of breast cancer comprising using for said diagnosis/prognosis at least one pair of primers as claimed in claim 13.

25. A method for diagnosis/prognosis of breast cancer comprising using for said diagnosis/prognosis at least one detection probe as claimed in claim 15.

26. A method for diagnosis/prognosis of breast cancer comprising using for said diagnosis/prognosis at least one detection probe as claimed in claim 16.

27. A kit for diagnosis/prognosis of breast cancer comprising at least one primer as claimed in claim 11.

28. A kit for diagnosis/prognosis of breast cancer comprising at least one pair of primers as claimed in claim 12.

29. A kit for diagnosis/prognosis of breast cancer comprising at least one pair of primers as claimed in claim 13.

30. A kit for diagnosis/prognosis of breast cancer comprising at least one detection probe as claimed in claim 15.

31. A kit for diagnosis/prognosis of breast cancer comprising at least one detection probe as claimed in claim 16.
